(19) United States
(12) Patent Application Publication
(10) Pub. No.: US 2022/0163379 A1
(43) Pub. Date: May 26, 2022

(54) RESONANCE FREQUENCY SHIFT SENSORS

(71) Applicant: Iowa State University Research Foundation, Inc., Ames, IA (US)

(72) Inventors: Nigel Forest Reuel, Ames, IA (US); Adam Russell Carr, Ames, IA (US)

(21) Appl. No.: 17/531,103

(22) Filed: Nov. 19, 2021

Related U.S. Application Data
(60) Provisional application No. 63/116,460, filed on Nov. 20, 2020.

(51) Int. Cl. G01H 13/00; C12Q 1/37

(52) U.S. Cl. CPC G01H 13/00; C12Q 1/37

(57) ABSTRACT

According to various aspects, a resonator includes a paper base. The paper base includes a channel bounded by least partially infused wax into the paper base. The resonator further includes an electronically conductive segment physically contacting the paper base. The resonator further includes a hydrogel component coating at least a portion of the electronically conductive segment.

### RELATED APPLICATIONS

This application claims priority to U.S. Provisional Patent Application No. 63/116,460 titled “RESONANCE FREQUENCY SHIFT SENSORS”, filed on Nov. 20, 2020, the contents of which are hereby incorporated by reference.

### STATEMENT OF GOVERNMENT SUPPORT

This invention was made with Government support under IIP2029532 awarded by the National Science foundation. The government has certain rights in this invention.

### BACKGROUND

Determining the presence and activity of an analyte can be useful in many different contexts. In some applications, this can be done indirectly by detecting the presence of byproducts of the reaction between an analyte and substrate. Indirect detection can be unreliable and potentially expensive. It may, therefore, be desirable to develop improved detection methods and assemblies.

### SUMMARY OF THE DISCLOSURE

According to various aspects, a resonator includes a paper base. The paper base includes a channel bounded by least partially infused wax into the paper base. The resonator further includes an electronically conductive segment physically contacting the paper base. The resonator further includes a hydrogel (e.g., gelatin) or enzyme substrate component coating at least a portion of the electronically conductive segment.

According to various aspects, a resonator includes a paper base. The paper base includes a channel bounded by least partially infused wax into the paper base. The resonator further includes an electronically conductive segment physically contacting the paper base. The electronically conductive segments comprises an Archimedean spiral profile and an inner diameter of the Archimedean spiral is greater than a pitch between adjacent arms of the Archimedean spiral The resonator further includes a hydrogel or enzyme substrate component coating at least a portion of the electronically conductive segment.

According to various aspects, a system includes a resonator that includes a paper base. The paper base includes a channel bounded by least partially infused wax into the paper base. The resonator further includes an electronically conductive segment physically contacting the paper base. The resonator further includes a hydrogel or enzyme substrate component coating at least a portion of the electronically conductive segment. The system further includes a resonator reader for detecting a resonant frequency and a shift in resonant frequency of the resonator.

According to various aspects, a method for detecting an analyte includes measuring a first resonant frequency of the resonator. The resonator includes a paper base. The paper base includes a channel bounded by least partially infused wax into the paper base. The resonator further includes an electronically conductive segment physically contacting the paper base. The resonator further includes a hydrogel or enzyme substrate component coating at least a portion of the electronically conductive segment. The method further includes exposing the hydrogel or enzyme substrate component to a solution and measuring a second resonant frequency of the resonator following exposure to the solution.

There are various advantages to using the systems and methods of the instant disclosure, some of which are unexpected. For example, according to some embodiments, inexpensive, flexible, wireless, resonant sensors can be rapidly fabricated. According to some embodiments, the frequency response window of the scattering parameter responses can be tuned by the resonator geometry. According to some embodiments, measuring the resonance frequency in a range of from about 1 to about 100 MHz provides a clean spectral background and sufficient signal penetration through a medium. According to some embodiments, the activity of a hydrolytic enzyme can be measured with a specific substrate to the enzyme and observing the degradation rate of the substrate as transduced wirelessly by a change in resonant frequency. According to some embodiments the resonators can be deloyed to provide real-time in situ measurements of enzymatic activity. According to some embodiments, the resonator can be a an open circuit as opposed to a closed circuit.

### BRIEF DESCRIPTION OF THE FIGURES

The drawings illustrate generally, by way of example, but not by way of limitation, various embodiments discussed in the present document.

FIG. 1 is a sectional view of a resonator.

FIG. 2 is a top view of an electronically conductive segment broken away from the resonator of FIG. 1.

FIG. 3 is a plan view of a resonator reader.

FIG. 4A is a graph showing the resonance frequency shift with a resonator having a channel oriented over a dead zone.

FIG. 4B is a graph showing the resonance frequency shift with a resonator having a channel oriented over only over an active zone.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “organic group” as used herein refers to any carbon-containing functional group. Examples can include an oxygen-containing group such as an alkoxy group, a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)O₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and. hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, or cycloalkylalkyl. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, hiphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, and the like; and R₃N wherein each R is independently selected, such as trialkylamines, dialkylarylamines, alkyldiarylamines, triarylamines, and the like. The term “amine” also includes ammonium ions as used herein.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “weight-average molecular weight” as used herein refers to M_{w}, which is equal to ΣMᵢ²nᵢ/ΣMᵢnᵢ, where nᵢ is the number of molecules of molecular weight Mᵢ. In various examples, the weight-average molecular weight can be determined using light scattering, small angle neutron scattering, X-ray scattering, and sedimentation velocity.

As used herein, the term “polymer” refers to a molecule having at least one repeating unit and can include copolymers.

The polymers described herein can terminate in any suitable way. In some embodiments, the polymers can terminate with an end group that is independently chosen from a suitable polymerization initiator, —H, —OH, a substituted or unsubstituted (C₁-C₂₀)hydrocarbyl (e.g., (C₁-C₁₀)alkyl or (C₆-C₂₀)aryl) interrupted with 0, 1, 2, or 3 groups independently selected from —O—, substituted or unsubstituted —NH—, and —S—, a poly(substituted or unsubstituted (C₁-C₂₀)hydrocarbyloxy), and a poly(substituted or unsubstituted (C₁-C₂₀)hydrocarbylamino

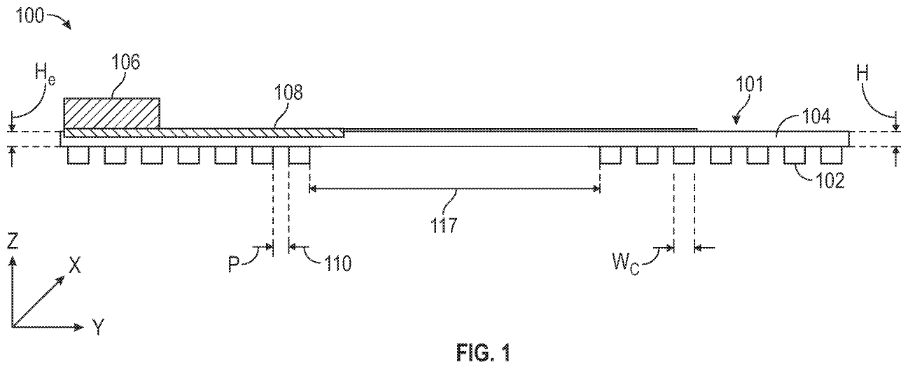

Described herein is a resonator for detecting analyte activity. In some aspects, the analyte can be an enzyme. When the analyte is an enzyme, the activity can be enzymatic activity, which can include the presence of an enzyme, a rate of reaction between an enzyme and a substrate, or other parameters. FIG. 1 is a sectional view of resonator system 100. Resonator system 100 includes resonator 101 including electronically conductive segment 102, paper base 104, substrate 106, and channel 108.

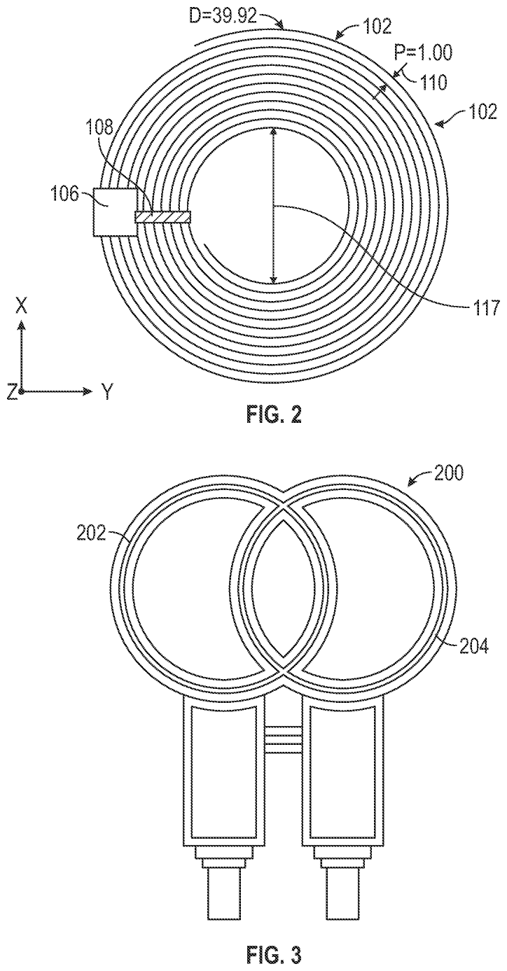

Electronically conductive segment 102 includes an electronically conductive metal. FIG. 2. is a top view of electronically conductive segment. As shown in FIG. 2, electronically conductive segment 102 includes a plurality of rings. Examples of suitable metals forming electronically conductive segment 102 include copper, silver, gold, aluminum, alloys thereof, or mixtures thereof. Electronically conductive segment 102 can be formed as a continuous segment or may include a plurality of discontinuous segments distributed through resonator 101. Electronically conductive segment 102 can take on any suitable shape or configuration. For example, electronically conductive segment 102 can be configured as a spiral in which adjacent portions or rings of electronically conductive segment 102 are spaced relative to each other defining a pitch therebetween, in some examples, the pitch can be constant across electronically conductive segment 102, thus, as shown, the spiral is an Archimedean spiral.

As shown in FIGS. 1 and 2, electronically conductive segment 102 is continuous. Electronically conductive segment 102 can have any suitable dimensions. For example, a total length of electronically conductive segment can be in a range of from about 5 mm to about 2000 mm, about 15 mm to about 40 mm, about 20 mm to about 25 mm, or less than, equal to, or greater than about 5 mm, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or about 2000 mm. In general, increasing the length of electronically conductive segment 102 decreases the resonance frequency of resonator 101. In embodiments of electronically conductive segment 102, such as that shown in FIGS. 1 and 2, where electronically conductive segment is a spiral the length refers to the total distances measured along electronically conductive segment from end to end.

A distance between opposed faces of adjacent portions of electronically conductive segment 102 is characterized as pitch 110. In some embodiments of conductive segment 102, pitch 110 is constant across all portions. In other embodiments, pitch 110 can be variable. In further embodiments, a first plurality of pitches 110 may be constant while a second plurality of pitches 110 may be variable. At each instance, pitch 110 can be in a range of from about 0.1 mm to about 10 mm, about 1 mm to about 3 mm, or less than, equal to, or greater than about 0.1 mm, 0.5, 1, 1.5, 2, 2.5, 3, 3.5, 4, 4.5, 5, 5.5, 6, 6.5, 7, 7.5, 8, 8.5, 9 9.5, or about 10 mm. Generally, increasing pitch 110 increases the resonance frequency of resonator 101.

As shown in FIG. 2 a gap 117 exists between the inner most segment of electronically conductive segment 102. A diameter of gap 117 is larger than pitch 110. Gap 117 is effectively an electronically dead zone that does not contribute to the resonant frequency shift.

As shown in FIGS. 1 and 2, resonator 101 has a circular profile. In other embodiments, however, resonator 101 can have any other suitable shape. For example, resonator 101 can have a polygonal profile such as a triangular shape, a square shape, a rectangular shape, a pentagonal shape, a hexagonal shape, a heptagonal shape, or an octagonal shape. A major dimension in the x-direction or y-direction that is perpendicular to electronically conductive segment 102 can be represented as a diameter or width of resonator 101. The major dimension can be in a range of from about 5 mm to about 100 mm, about 15 mm to about 60 mm, or less than, equal to, or greater than about 5 mm, 10, 15, 20, 25, 30, 35, 40, 45, 50, 55, 60, 65, 70, 75, 80, 85, 90, 95, or about 100 mm.

A height or thickness, measured in the z-direction, of electronically conductive segment 102 can be set to any value. For example, a thickness of electronically conductive segment 102 can be in a range of from about 10 μm to about 100 μm, about 20 μm to about 40 μm, or less than, equal to, or greater than about 10 μm, 15, 20, 25, 30, 35, 40, 45, 50, 55, 60, 65, 70, 75, 80, 85, 90, 95, or about 100 μm. The thickness of electronically conductive segment 102 can affect the resonance frequency of resonator 101. Increasing the thickness of electronically conductive segment 102 too much, however, can result in resonator 101 being too thick for certain applications.

Paper base 104 is located between electronically conductive segment 102 and substrate 106. Paper base 104 includes one or more channels 108. Channel 108 is disposed at or near substrate 106. Channel 108 can run towards gap 117 from any location on resonator 101. As shown, channel 108 spans between a location proximate substrate 106 to gap 117. Channel 108 is formed by infusing wax into paper base 104. The infused wax creates the walls of channel 108. Channel 108 is shown as having a straight profile. However, in further aspects, channel 108 can have any other profile such as an undulating profile. In some aspects, one portion of channel 108 can be substantially straight, while another portion is undulating. Although only one channel 108 is shown, in some aspects a plurality of channels 108 can be included.

Substrate 106 is in direct contact with channel 108. Substrate 106 can be a substrate of one or more enzymes. For example, substrate 106 can be a substrate of a hydrolase enzyme (alternatively known as a EC 3 enzyme). The hydrolase can be classified by the bond it acts upon. For example, the hydrolase can be chosen from an esterase, nuclease, phosphodiesterase, lipase, phosphatase, DNA glycosylase, glycoside hydrolase, proteases, peptidase, acid anhydride hydrolase, GTPase, GTPase, alcalase, or mixtures thereof. The enzyme can be present in system 100 or may be an external component that interacts with system 100. Additional enzymes may include a ligase and a lyase. Substrate 106 can be a freestanding structure on channel 108.

Substrate 106 can be a substrate for any predetermined enzyme. That is, substrate 106 can be a substrate of the enzyme to which resonator system 100 is configured to detect enzymatic activity. In some embodiments, substrate 106 is a substrate of a hydrolase enzyme. As an example, substrate 106 can be a hydrogel substrate Where the substrate 106 is a substrate of a hydrolase, substrate can include a bond that is hydrolyzable by the hydrolase. Examples of such bonds can include an ester bond, a glycosylic bond, an ether bond, a peptide bond, an acid anhydride bond, a halide bond, a phosphorous-sulfur bond, a sulfur-sulfur bond, a carbon-phosphorous bond, a carbon-sulphur bond, or a combination thereof. Examples of suitable substrates include a bovine serum albumin, citrus pectin, carboxymethyl cellulose, hydrogel, polylactic acid, or a mixture thereof. In some examples, the hydrogel can be degraded upon exposure to a certain temperature or range of temperatures, a certain pH or range of pHs, the electric field of an environment, or the ionic strength of the environment to which it is exposed.

Resonator system 100 can further include resonator reader 200. FIG. 3 is a plan view of resonator reader 200. As shown in FIG. 3 resonator reader 200 includes first electronically conductive loop 202 and second electronically conductive loop 204. Resonator reader 200 further includes first connector 206 and second connector 208. First electronically conductive loop 202 and second electronically conductive loop 206 can independently include an electronically conductive metal such as copper, silver, gold, aluminum, alloys thereof, or mixtures thereof. A diameter of each of loops 202 and 204 can independently range from about 5 mm to about 100 mm, about 15 mm to about 60 mm, or less than, equal to, or greater than about 5 mm, 10, 15, 20, 25, 30, 35, 40, 45, 50, 55, 60, 65, 70, 75, 80, 85, 90, 95, or about 100 mm. As shown in FIG. 3, loops 202 and 204 overlap. A distance between overlapping regions of reader 200 can be in a range of from about 5 mm to about 100 mm, about 15 mm to about 60 mm, or less than, equal to, or greater than about 5 mm, 10, 15, 20, 25, 30, 35, 40, 45, 50, 55, 60, 65, 70, 75, 80, 85, 90, or about 95.

Connectors 206 and 208 can connect resonator reader 200 to a component such as a vector network analyzer. The vector network analyzer can in turn be connected to a computer. The connection between the vector network analyzer and the computer can be through a wire or an antenna. Loops 202 and 204 as well as connectors 206 and 208 are at least partially enclosed by a dielectric material. Examples of suitable dielectric materials include a polyimide, a bismaleimide-triazine (BT) resin, an epoxy resin, a polyurethane, a benzocyclobutene (BCB), a high-density polyethylene (HDPE), and combinations thereof.

Resonator reader 200 can be positioned substantially in line with resonator 101. A distance between resonator reader 200 and resonator 101 can be varied to improve performance. For example, a distance between resonator reader 200 and resonator 101 can be in a range of from about 1 mm to about 10 cm.

Resonator system 100 is described as including one resonator 101 and one resonator reader 200. However, in further embodiments resonator system 100 can include any plural number of resonators 101 and readers 200. In embodiments that include multiple resonators, each resonator can be designed to have a different initial resonant frequency. This can be accomplished by varying any parameter such as respective lengths of electronically conductive segments 102 or altering pitches 110. The resonators can also differ by the composition of the respective substrates. For example, a substrate of one substrate can be a substrate of a first enzyme while another substrate on another resonator may be a substrate for a different enzyme.

In operation, detecting enzymatic activity or the presence of an enzyme using resonator system 100 includes measuring a first resonance frequency of resonator 101. The first resonance frequency is the resonance frequency of resonator 101 before substrate 106 is contacted with an enzyme. When the enzyme is contacted with substrate 106, substrate 106 is consumed. As substrate 106 is consumed, the resonance frequency of resonator 101 changes. Thus, if a second resonance frequency of resonator 101 is measured that is different than the first resonance frequency the presence of the enzyme can be confirmed. By measuring the rate of change of the resonance frequency it is possible to monitor the rate of reaction between substrate 106 and the enzyme. At least one of the first resonance frequency and the second resonance frequency can be in a range of from about 1 MHz to about 500 MHz, about 1 MHz to about 100 MHz, or less than, equal to, or greater than about 1 MHz, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or about 500 MHz.

To help create a high signal to noise ratio, consumed substrate 106 is passed through channel 108 to gap 107. Passing along the consumed substrate 106 helps to prevent the consumed substrate from interfering with electronically conductive segment 102 and therefore obscuring the detection of the resonance frequency shift.

In embodiments where resonator system 100 includes multiple resonators 101, each resonator 101 may have different substrates 106 for different enzymes. Each resonator can be configured to have a different first resonance frequency. Therefore, specific substrates 106 for predetermined enzymes can be paired with resonators having specific known first resonance frequencies. If the resonance frequency of one of resonators 101 begins to change then the presence of a specific enzyme and the absence of another can be confirmed.

Resonator system 100 can be deployed in many different mediums to detect enzymatic activity. For example, resonator system 100 can be placed in soil, fabric, or a tank. If placed in soil, resonator system 100 can detect the presence of certain harmful or beneficial enzymes that can impact the viability of crops. If placed in a tank, resonator system 100 can be used to detect enzymes in a storage tank used for example to store chemicals, beverages, medicine, or drinking water. The tank can also be a component of a bioreactor. The presence of the enzyme may indicate that the solution stored in the tank is not safe for consumption. Alternatively, if the presence of a certain enzyme is desirable, then the levels of the enzyme can be monitored. If resonator system 100 is placed in fabric, then it can be possible to determine whether the fabric is exposed to a biological agent. For example, resonator system 100 can be placed in a garment of a military member or first responder to allow them to know in real time whether they have been exposed to a biological agent.

Resonator system 100 can be assembled according to any suitable method. For example, an assembly including any of the electronically conductive metals described herein coated to paper base 104. Portions of paper base 104 between segments of the electronically conductive metal can be cut away. Channel 108 is formed by infusing wax into the paper. Substrate 107 is then placed in contact with channel 108.

### EXAMPLES

Various embodiments of the present disclosure can be better understood by reference to the following Examples, which are offered by way of illustration. The present disclosure is not limited to the Examples given herein.

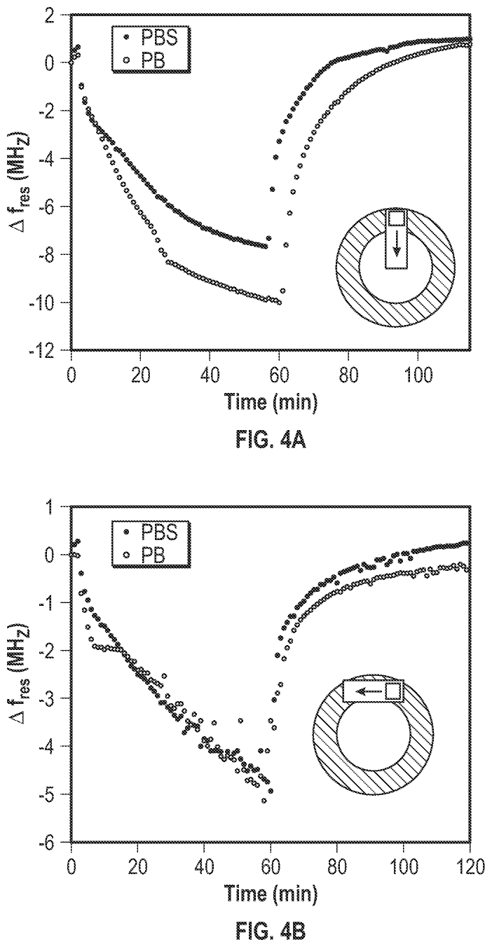

Two resonators are designed one resonator, as shown in FIG. 4A, includes a channel oriented to be in communication with both the active zone and dead zone of the resonator. The other resonator, as shown in FIG. 4B, has the channel only oriented over the active zone. Each of the resonator contractions were fitted with a hydrogel or enzyme substrate and exposed to a respective PBS buffer and bacterial protease. The bacterial protease was capable of digesting the hydrogel or enzyme substrate. As shown in FIGS. 4A and 4B, the resonator of FIG. 4A shows greater sensitivity than the resonator of FIG. 4B. This shows that being able to route digested substrate to a dead zone and away from the active zone can improve the sensitivity of the resonator.

### Additional Aspects

The following exemplary aspects are provided, the numbering of which is not to be construed as designating levels of importance:

Aspect 1 provides a resonator comprising:

a paper base, comprising
a channel bounded by least partially infused wax into the paper base;

an electronically conductive segment physically contacting the paper base; and

a hydrogel or enzyme substrate component coating at least a portion of the electronically conductive segment.

Aspect 2 provides the resonator of Aspect 1, wherein the channel is a first channel and the paper base comprises a second channel.

Aspect 3 provides the resonator of any one of Aspects 1 or 2, wherein the channel comprises a straight profile.

Aspect 4 provides the resonator of any one of Aspects 1 or 3, wherein the channel comprises an undulating profile.

Aspect 5 provides the resonator of any one of Aspects 1-4, wherein the channel comprises a combination of a straight profile and an undulating profile.

Aspect 6 provides the resonator of any one of Aspects 1-5, wherein the channel is in physical contact with the hydrogel or enzyme substrate component.

Aspect 7 provides the resonator of any one of Aspects 1-6, the channel terminates at a location distal to the electronically conductive component.

Aspect 8 provides the resonator of any one of Aspects 1-7, wherein a profile of the conductive segment is an Archimedean spiral comprising one or more rings spaced relative to one another.

Aspect 9 provides the resonator of Aspect 8, wherein a pitch between the rings is constant across a first portion of the spiral.

Aspect 10 provides the resonator of Aspect 9, wherein a pitch defined by a space between an innermost ring is greater than the pitch between rings in the first portion.

Aspect 11 provides the resonator of any one of Aspects 9 or 10, wherein the pitch is in a range of from about 0.1 mm to about 10 mm.

Aspect 12 provides the resonator of any one of Aspects 9-11, wherein the pitch is in a range of from about 1 mm to about 3 mm.

Aspect 13 provides the resonator of any one of Aspects 1-12, wherein a thickness of the electronically conductive segment is in a range of from about 0.001 mm to about 5 mm.

Aspect 14 provides the resonator of any one of Aspects 1-13, wherein a thickness of the electronically conductive segment is in a range of from about 0.5 mm to about 1.5 mm.

Aspect 15 provides the resonator of any one of Aspects 1-14, wherein the electronically conductive segment comprises a metal.

Aspect 16 provides the resonator of any one of Aspects 1-15, wherein the electronically conductive segment comprises copper, silver, gold, aluminum, gallium, indium, alloys thereof, or mixtures thereof.

Aspect 17 provides the resonator of any one of Aspects 1-16, wherein the electronically conductive segment comprises a continuous segment.

Aspect 18 provides the resonator of any one of Aspects 1-17, wherein the electronically conductive segment comprises discontinuous segments.

Aspect 19 provides the resonator of any one of Aspects 1-18, wherein the hydrogel or enzyme substrate component coats from about 10 percent surface area to about 100 percent surface area of the electronically conductive segment.

Aspect 20 provides the resonator of any one of Aspects 1-19, wherein the hydrogel or enzyme substrate component coats from about 20 percent surface area to about 33 percent surface area of the electronically conductive segment.

Aspect 21 provides the resonator of any one of Aspects 1-20, wherein the hydrogel or enzyme substrate component is discontinuous.

Aspect 22 provides the resonator of any one of Aspects 1-21, wherein the hydrogel or enzyme substrate component is a substrate of a hydrolytic enzyme.

Aspect 23 provides the resonator of Aspect 22, wherein the hydrolytic enzyme comprises a protease

Aspect 24 provides a resonator comprising:

a paper base, comprising
a channel bounded by wax that is at least partially infused into the paper base;

an electronically conductive segment contacting the paper base, wherein the electronically conductive segments comprises an Archimedean spiral profile and an inner meter of the Archimedean spiral is greater than a pitch between adjacent arms of the Archimedean spiral; and

a hydrogel or enzyme substrate component coating at least a portion of the electronically conductive segment and positioned over at least a portion of the channel.

Aspect 25 provides a system comprising the resonator of any one of Aspects 1-24, and further comprising a resonator reader for detecting a resonant frequency and a shift in resonant frequency of the resonator.

Aspect 26 provides the system of Aspect 25, wherein the resonator reader is positioned in-line with the resonator.

Aspect 27 provides the system of any one of Aspects 25 or 26, further comprising a vector network analyzer connected to the resonator reader.

Aspect 28 provides the system of Aspect 27, wherein the vector network analyzer is further coupled to a computer.

Aspect 29 provides the system of any one of Aspects 25-28, further comprising an antenna coupled to the resonator.

Aspect 30 provides the system of any one of Aspects 25-29, wherein the resonator is a first resonator and the system further comprises a second resonator.

Aspect 31 provides the system of Aspect 30, wherein a resonant frequency of the first resonator is different than a resonant frequency of the second resonator.

Aspect 32 provides a method of detecting an analyte, the method comprising:

measuring a first resonant frequency of the resonator according to any one of Aspects 1-31;

exposing the hydrogel or enzyme substrate component to a solution; and

measuring a second resonant frequency of the resonator following exposure to the solution.

Aspect 33 provides the method of Aspect 32, wherein the second resonant frequency is less than the first resonant frequency.

Aspect 34 provides the method of any one of Aspects 32 or 33, wherein at least one of the first resonant frequency and the second resonant frequency are in a range of from about 1 MHz to about 500 MHz.

Aspect 35 provides the method of any one of Aspects 32-34, wherein at least one of the first resonant frequency and the second resonant frequency are in a range of from about 10 MHz to about 100 MHz.

Aspect 36 provides the method of any one of Aspects 32-35, wherein the solution further comprises the analyte.

Aspect 37 provides a method of making the resonator according to any one of Aspects 1-36, the method comprising:

printing the electronically conductive segment to the paper base to form a pattern therein;

infusing portions of the paper base with wax;

removing a portion of the paper base between adjacent electronically conductive segments; and

applying the hydrogel or enzyme substrate component to a portion of the electronically conductive segment.

What is claimed is:

1. A resonator comprising:
a paper base, comprising
a channel bounded by least partially infused wax into the paper base;
an electronically conductive segment physically contacting the paper base; and
a hydrogel or enzyme substrate component coating at least a portion of the electronically conductive segment.

2. The resonator of claim 1, wherein the channel is a first channel and the paper base comprises a second channel.

3. The resonator of claim 1, wherein the channel comprises a combination of a straight profile and an undulating profile.

4. The resonator of claim 1, wherein the channel is in physical contact with the hydrogel or enzyme substrate component.

5. The resonator of claim 1, the channel terminates at a location distal to the electronically conductive component.

6. The resonator of claim 1, wherein a profile of the conductive segment is an Archimedean spiral comprising one or more rings spaced relative to one another.

7. The resonator of claim 1, wherein the electronically conductive segment comprises a metal.

8. The resonator of claim 1, wherein the electronically conductive segment comprises copper, silver, gold, aluminum, gallium, indium, alloys thereof, or mixtures thereof.

9. The resonator of claim 1, wherein the electronically conductive segment comprises a continuous segment.

10. The resonator of claim 1, wherein the hydrogel or enzyme substrate component coats from about 10 percent surface area to about 100 percent surface area of the electronically conductive segment.

11. The resonator of aim 1, wherein the hydrogel or enzyme substrate component is discontinuous.

12. The resonator of claim 1, wherein the hydrogel or enzyme substrate component s a substrate of a hydrolytic enzyme.

13. The resonator of claim 12, wherein the hydrolytic enzyme comprises a protease

14. A resonator comprising:
a paper base, comprising
a channel bounded by wax that is at least partially infused into the paper base;
an electronically conductive segment contacting the paper base, wherein the electronically conductive segments comprises an Archimedean spiral profile and an inner diameter of the Archimedean spiral is greater than a pitch between adjacent arms of the Archimedean spiral; and
a hydrogel or enzyme substrate component coating at least a portion of the electronically conductive segment and positioned over at least a portion of the channel.

15. A system comprising the resonator of claim 1, and further comprising a resonator reader for detecting a resonant frequency and a shift in resonant frequency of the resonator.

16. The system of claim 15, wherein the resonator is a first resonator and the system further comprises a second resonator.

17. The system of claim 16, wherein a resonant frequency of the first resonator is different than a resonant frequency of the second resonator.

18. A method of detecting an analyte, the method comprising:
measuring a first resonant frequency of the resonator of claim 1;
exposing the hydrogel or enzyme substrate component to a solution; and
measuring a second resonant frequency of the resonator following exposure to the solution.

19. The method of claim 18, wherein the second resonant frequency is less than the first resonant frequency.

20. A method of making the resonator of claim 1, the method comprising:
printing the electronically conductive segment to the paper base to form a pattern therein;
infusing portions of the paper base with wax;
removing a portion of the paper base between adjacent electronically conductive segments; and
applying the hydrogel or enzyme substrate component to a portion of the electronically conductive segment.
